(19) United States
(12) Patent Application Publication
(10) Pub. No.: US 2025/0049685 A1
(43) Pub. Date: Feb. 13, 2025

(54) COLLAGEN LOTION FORMULATIONS AND METHODS OF USE THEREOF

(71) Applicant: Wellbeam Consumer Health Intermediate, LLC, San Rafael, CA (US)

(72) Inventor: Jalshree Trivedi, Fremont, CA (US)

(73) Assignee: Wellbeam Consumer Health Intermediate, LLC, San Rafael, CA (US)

(21) Appl. No.: 18/802,452

(22) Filed: Aug. 13, 2024

Related U.S. Application Data
(60) Provisional application No. 63/519,259, filed on Aug. 13, 2023.

(51) Int. Cl. A61K 8/65; A61Q 19/08

(52) U.S. Cl. CPC A61K 8/65; A61Q 19/08; A61K 2800/51; A61K 2800/522; A61K 2800/524; A61K 2800/5922; A61K 2800/85

(57) ABSTRACT

Cosmetic compositions for skincare with a unique blend of plant-derived ingredients to reduce prevent, reduce, and treat damage to the skin. Also provided are methods of improving one or more skin conditions by applying the topical compositions disclosed herein.

### CROSS REFERENCE TO RELATED APPLICATION

This application claims priority to U.S. Provisional Patent Application Ser. No. 63/519,259, filed Aug. 13, 2023, and titled “COLLAGEN LOTION FORMULATIONS AND METHODS OF USE THEREOF,” which is incorporated by reference herein in its entirety.

### BACKGROUND

### 1. Field

The present disclosure relates to topical cosmetic compositions for skincare.

### 2. Background

The skin is the largest organ in the human body and consists essentially of two primary layers—the epidermis and the dermis. The dermis consists largely of the protein collagen, which forms a network of cross-linked fibers made up of collagen, fibroblast, and reticular and elastic fibers, providing a framework for blood vessels and cell growth. Because it is the primary component of the dermis, collagen acts as the support structure for the skin. The health and stability of collagen is a critical factor in determining the contour, wrinkles and lines in the skin. There are 28 types of collagen in the body, among which Type I and Type Ill are the most prominent collagen types in dermis. Further, elastin is a protein that works with collagen to support the epidermis of the skin and is important in maintaining the flexibility and stretchability of the skin.

Different factors affect natural decline of collagen and elastin protein production including a range of environmental assaults such as oxidative stress, ultraviolet (UV) rays, high energy visible light (violet-blue) emitted by devices such as TV/computer/smartphone screens, as well as harmful chemical agents found in airborne pollution such as smog and cigarette smoke. Additionally, skin is also subject to structural damage due to aging and other biological factors. Aging is a natural process which can be divided into intrinsic and extrinsic aging. Intrinsic aging occurs with the passage of time, determined genetically, with slow tissue deterioration, hormonal status, and metabolic reactions, such as oxidative stress. Extrinsic aging is caused by external factors, such as chronic sun exposure or cigarette smoke. It is therefore desirable to apply formulations that can reduce structural damages, improve collagen production, and adequately nourish and hydrate the skin.

There is a continuing need to provide compositions and methods of treatment that can protect the skin barrier, and smoothen, nourish, and improve firmness of the skin. These and other improvements over the art are provided by the present disclosure.

### SUMMARY

In some aspects, the present disclosure encompasses a topical composition, comprising: vegan collagen peptide, Chlorella vulgaris extract, at least one ceramide, at least one excipient, and at least one solvent.

In some aspects, the vegan collagen peptide is present in the composition at a range of about 0.01% (w/w) to about 1% (w/w). In some aspects, the vegan collagen peptide is Sh-Polypeptide-121.

In some aspects, the Chlorella vulgaris extract is present in the composition at a range of about 0.01% (w/w) to about 1% (w/w).

In some aspects, the topical composition comprises at least one ceramide wherein the ceramide is Ceramide NP, Ceramide AP, Ceramide EOP, or any combination thereof. In some aspects, the ceramide is present in the composition at a range of about 0.01% (w/w) to about 3% (w/w).

In some aspects, the at least one excipient is present in the composition at about 0.1% (w/w) to about 90% (w/w). In some aspects, the at least one excipient is polysorbate 20, cetearyl alcohol, glycerin, sodium lauroyl lactylate, carbomer, xanthan gum, or any combination thereof.

In some aspects, the at least one solvent is present in the composition at about 1% (w/w) to about 90% (w/w). In some aspects, the at least one solvent is water.

In some aspects, the composition further comprises at least one skin conditioner and/or at least one emollient. In some aspects, the at least one skin conditioner and/or at least one emollient is caprylic/capric triglyceride, coconut alkanes, glyceryl stearate, Helianthus annuus seed oil, Aloe barbadensis leaf juice, Cocos nucifera oil, coco-caprylate/caprate, phytosphingosine, cholesterol, ethylhexylglycerin, or any combination thereof. In some aspects, the at least one skin conditioner and/or at least one emollient is present in the composition at about 1% (w/w) to about 10% (w/w).

In some aspects, the composition further comprises an antioxidant. In some aspects, the antioxidant is Tocopheryl acetate. In some aspects, the antioxidant is present at the composition at about 0.01% (w/w) to about 1% (w/w).

In some aspects, the composition further comprises at least one preservative. In some aspects, the at least one preservative is phenoxyethanol, potassium sorbate, or any combination thereof. In some aspects, the at least one preservative is present in the composition at about 0.01% (w/w) to about 1% (w/w).

In some aspects, the composition further comprises at least one buffer. In some aspects, the at least one buffer is sodium hydroxide. In some aspects, the at least one buffer is present in the composition at about 0.01% (w/w) to about 1% (w/w).

In some aspects, the topical composition further comprises at least one chelator. In some aspects, the chelator is sodium phytate. In some aspects, the chelator is present in the composition at about 0.01% (w/w) to about 1% (w/w).

In some aspects, the topical composition further comprise oils, fats, thickening agents, coloring agents, fragrance, or any combination thereof.

In further aspects, provided herein is a topical composition comprising: water, caprylic/capric triglyceride, coconut alkanes, glyceryl stearate, polysorbate 20, Helianthus annuus seed oil, Aloe barbadensis leaf juice, cetearyl alcohol, Cocos nucifera oil, glycerin, coco-caprylate/caprate, sodium phytate, Chlorella vulgaris extract, vegan collagen peptide, ceramide NP, ceramide AP, ceramide EOP, phytosphingosine, cholesterol, sodium lauroyl lactylate, carbomer, ethylhexylglycerin, xanthan gum, phenoxyethanol, sodium hydroxide, tocopheryl acetate, potassium sorbate, or any combination thereof.

In certain aspects, the disclosure encompasses a topical composition comprising:
Water: about 1% (w/w) to about 90% (w/w);
Caprylic/Capric triglyceride: about 1% (w/w) to about 10% (w/w);
Coconut alkanes: about 1% (w/w) to about 10% (w/w);
Glyceryl stearate: about 1% (w/w) to about 10% (w/w);
Polysorbate 20: about 1% (w/w) to about 10% (w/w);
Helianthus annuus seed oil: about 1% (w/w) to about 10% (w/w);
Aloe barbadensis leaf juice: about 1% (w/w) to about 10% (w/w);
Cetaryl alcohol: about 1% (w/w) to about 10% (w/w);
Cocos nucifera oil: about 1% (w/w) to about 10% (w/w);
Glycerin: about 0.01% (w/w) to about 1% (w/w);
Coco-Caprylate/Caprate: about 0.01% (w/w) to about 1% (w/w);
Sodium Phytate: about 0.01% (w/w) to about 1% (w/w);
Chlorella vulgaris extract: about 0.01% (w/w) to about 1% (w/w);
sh-Polypeptide-121: about 0.01% (w/w) to about 1% (w/w);
Ceramide NP: about 0.01% (w/w) to about 1% (w/w);
Ceramide AP: about 0.01% (w/w) to about 1% (w/w);
Ceramide EOP: about 0.01% (w/w) to about 1% (w/w);
Phytosphingosine: about 0.01% (w/w) to about 1% (w/w);
Cholesterol: about 0.01% (w/w) to about 1% (w/w);
Sodium lauroyl lactylate: about 0.01% (w/w) to about 1% (w/w);
Carbomer: about 0.01% (w/w) to about 1% (w/w);
Ethylhexylglycerin: about 0.01% (w/w) to about 1% (w/w);
Xanthan gum: about 0.01% (w/w) to about 1% (w/w);
Phenoxyethanol: about 0.01% (w/w) to about 1% (w/w);
Sodium hydroxide: about 0.01% (w/w) to about 1% (w/w);
Tocopheryl acetate: about 0.01% (w/w) to about 1% (w/w);
Potassium sorbate: about 0.01% (w/w) to about 1% (w/w).

In some aspects, the topical composition disclosed herein is administered to the skin of a subject to prevent, reduce, or treat fine lines, aging of the skin cells, crow's feet, wrinkles, moisture loss, hyperpigmentation, uneven skin tone, under eye circles, uneven skin pigmentation, crepe skin, collagen loss, elastin loss, oxidation, skin redness, roughness, free radical formation, UV damage, photo-damage, blue light damage, or any combination thereof.

In some aspects, the disclosed topical composition is administered to the skin of a subject to enhance collagen production, elastin production, skin immunity, hydration, clarity, radiance, passage for absorption, skin brightness, healthy glow, even tone, elasticity, firmness, smoothness, or any combination thereof.

In some aspects, the disclosed topical composition is in the form of a solids, liquids, suspensions, semisolids (such as creams, gels, pastes or “sticks”), powders or finely dispersed liquids such as sprays or mists. In certain aspects, the disclosed topical composition is in the form of a lotion.

Further provided herein is a method of improving the condition of the skin of a subject in need thereof comprising, applying to the skin of the subject the topical composition disclosed herein. In some aspects, the subject is a human.

In some aspects, further provided herein is a kit for skincare comprising the topical composition of any one of the preceding claims, and instructions for use.

### DETAILED DESCRIPTION

The description is intended to describe aspects and aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

The present disclosure is based at least in part on the unexpected result that a blend of plant-based ingredients comprising a vegan collagen peptide, Chlorella vulgaris extract, one or more ceramides, and/or one or more excipients increase skin firmness, lighten under-eye circles and improve collagen production in the skin. The composition can comprise a vegan collagen peptide, a natural collagen alternative, which can improve collagen type I production, Chlorella vulgaris extract, which can restore firmness of skin and protects loss of collagen and elastin in skin cells, and at least one ceramide which is essential in maintaining the skin's structure and for improving skin barrier function. In some aspects, the composition can be formulated with other ingredients to provide additional properties, for example, suitable consistency, desired applicability profile, and other skin care effects.

### I. Compositions

In some aspects, the present disclosure encompasses a topical composition comprising a vegan collagen peptide, Chlorella vulgaris extract, at least one ceramide, at least one excipient, and/or at least one solvent. More specifically, the composition comprises a vegan collagen peptide, Chlorella vulgaris extract, at least one ceramide, at least one excipient, at least one solvent, at least one skin conditioner and/or an emollient, at least one antioxidant, at least one preservative, at least one buffer, and/or at least one chelator. In some aspects, the ingredients used in the disclosed composition are natural, are derived from plants, are vegan, and/or are derived from non-GMO or other sustainable sources.

### Vegan Collagen Peptide

In some aspects, the topical composition comprises a natural collagen alternative. In some aspects, the natural collagen alternative is a vegan collagen peptide. In some aspects, the vegan collagen peptide in the topical composition is a plant or marine derived collagen, for example Type 21 collagen peptide. In one aspect, the vegan collagen is a vegan marine collagen. In some aspects, the Type 21 collagen peptide is a sh-Polypeptide-121. In such aspects, the topical composition comprises a sh-Polypeptide-121. sh-Polypeptide-121 is a synthetic single-chain recombinant peptide derived from human collagen type 21 (also called collagen alpha-1 and encoded by the COL21A1 gene). In one aspect, the sh-Polypeptide 121 is present as composition comprising sh-Polypeptide 121 and potassium sorbate. Vegan collagen peptide can be isolated and purified from any suitable source, using known methods in the art, or can be commercially obtained (for e.g., Geltor HumaColl™21, Geltor Elastapure® Powder).

In some aspects, vegan collagen peptide (e.g., sh-Polypeptide-121) in the disclosed topical composition reduces one or more of signs of aging on skin, skin redness, fine lines, wrinkles, or any combination thereof. In some aspects, vegan collagen peptide (e.g., sh-Polypeptide-121) in the disclosed topical composition enhances one or more of firmness, elasticity, collagen production, skin brightness, softness, smoothness, or any combination thereof. In further aspects, vegan collagen peptide (e.g., sh-Polypeptide-121) in the disclosed topical composition enhances collagen and/or elastin production in the skin.

In some aspects, the topical composition comprises a vegan collagen peptide in the amount of about 0.01% (w/w) to about 1% (w/w). In some aspects, the topical composition comprises a vegan collagen peptide in the amount of about 0.01% (w/w) to about 1% (w/w). In some aspects, the topical composition comprises a vegan collagen peptide in the amount of about 0.01% (w/w) to about 0.05% (w/w), or about 0.3% (w/w) to about 0.1%, or about 0.05% (w/w) to about 0.1% (w/w), or about 0.1% (w/w) to about 0.5% (w/w), or about 0.5% (w/w) to about 1% (w/w). In some aspects, the topical composition comprises a vegan collagen peptide at 0.1% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of a vegan collagen peptide. In some aspects, a vegan collagen peptide is present in the composition at about 0.03% (w/w). In some aspects, a vegan collagen peptide is present in the composition at about 0.01% (w/w). In some aspects, a vegan collagen peptide is present in the composition at about less than 1% (w/w). In some aspects, the topical composition comprises sh-Polypeptide-121 at an amount of about 0.03% (w/w). In some aspects, sh-Polypeptide-121 is present in the topical composition at about 0.01% (w/w). In some aspects, sh-Polypeptide-121 is present in the topical composition at about less than 1% (w/w).

Chlorella vulgaris Extract

In some aspects, the topical composition comprises a green algal extract. In some aspects, the green algal extract present in the topical composition is a Chlorella vulgaris extract. Chlorella vulgaris extract is high in anti-oxidants, can protect collagen and elastin degradation in the skin, can restore skin firmness and can combat aging. Chlorella vulgaris extract can reduce the appearance of under-eye dark circles, uneven skin tone, and uneven pigmentation. Chlorella vulgaris extract can also provide smoothing effect on the skin. Chlorella vulgaris extract can be obtained from any suitable source for e.g., commercially, from Barnet (Chlorellagen DP).

In some aspects, Chlorella vulgaris extract in the disclosed topical composition enhances skin firmness, smoothness, tonicity, collagen production, elastin synthesis, or any combination thereof. In some aspects, Chlorella vulgaris extract in the disclosed topical composition can decrease skin pigmentation, and can slow down the aging process of skin cells. In some aspects, Chlorella vulgaris extract in the disclosed topical composition can lighten undereye dark circles.

In some aspects, the topical composition comprises Chlorella vulgaris extract in the amount of about 0.01% (w/w) to about 1% (w/w). In some aspects, the topical composition comprises Chlorella vulgaris extract in the amount of about 0.01% (w/w) to about 1% (w/w). In some aspects, the topical composition comprises Chlorella vulgaris extract in the amount of about 0.01% (w/w) to about 0.05% (w/w), or about 0.3% (w/w) to about 0.1%% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises Chlorella vulgaris extract at 0.1% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of Chlorella vulgaris extract. In some aspects, Chlorella vulgaris extract is present in the topical composition at about 1% (w/w). In some aspects, Chlorella vulgaris extract is present in the topical composition at about 0.01% (w/w). In some aspects, Chlorella vulgaris extract is present in the topical composition at about less than 1% (w/w).

### Ceramide

In some aspects, the topical composition comprises at least one ceramide. In some aspects, the at least one ceramide present in the topical composition is Ceramide NP, Ceramide AP, Ceramide EOP, or any combination thereof. In some aspects, the topical composition comprises Ceramide NP. In some aspects, the topical composition comprises Ceramide AP. In some aspects, the topical composition comprises Ceramide EOP. In some aspects, the topical composition comprises Ceramide NP, Ceramide AP, and Ceramide EOP.

Ceramides are epidermal lipids that are essential for skin barrier function. Ceramides acts as a glue that holds the skin cells together and are essential in maintaining the skin's structure responsible for the water permeability barrier function of the skin. Imbalance in ceramides can weaken the skin barrier and can cause skin diseases. The ceramides can be obtained from any suitable source for e.g., using chemical synthesis or commercially.

In some aspects, the at least one ceramide in the disclosed topical composition enhances skin smoothness, hydration, or any combination thereof. In some aspects, the at least one ceramide in the disclosed topical composition can decrease wrinkle formation, skin dryness and can slow down the aging process of skin cells. In some aspects, the at least one ceramide in the disclosed topical composition can lighten undereye dark circles. In some aspects, the at least one ceramide in the disclosed topical composition can protect and/or restore skin's barrier.

In some aspects, the topical composition comprises at least one ceramide in the amount of about 0.01% to about 3%. In some aspects, the topical composition comprises at least one ceramide in the amount of about 0.01% (w/w) to about 1% (w/w). In some aspects, the topical composition comprises at least one ceramide in the amount of about 0.01% (w/w) to about 0.05% (w/w), or about 0.3% (w/w) to about 0.1%, or about 0.05% (w/w) to about 0.1% (w/w), or about 0.1% (w/w) to about 0.5% (w/w), or about 0.5% (w/w) to about 1% (w/w), or about 1% (w/w) to about 1.5% (w/w), or about 1.5% (w/w) to about 2% (w/w), or about 2% (w/w) to about 3% (w/w). In some aspects, the topical composition comprises at least one ceramide at 0.1% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), or 3% (w/w) of at least one ceramide. In some aspects, at least one ceramide is present in the topical composition at about 1% (w/w). In some aspects, at least one ceramide is present in the topical composition at about 0.01% (w/w). In some aspects, at least one ceramide is present in the topical composition at about less than 1% (w/w). In some aspects, the topical composition comprises ceramide NP at about 1% (w/w). In some aspects, the topical composition comprises ceramide NP at about 0.01% (w/w). In some aspects, the topical composition comprises ceramide NP at about less than 1% (w/w). In some aspects, the topical composition comprises ceramide AP at about 1% (w/w). In some aspects, the topical composition comprises ceramide AP at about 0.01% (w/w). In some aspects, the topical composition comprises ceramide AP at about less than 1% (w/w). In some aspects, the topical composition comprises ceramide EOP at about 1% (w/w). In some aspects, the topical composition comprises ceramide EOP at about 0.01% (w/w). In some aspects, the topical composition comprises ceramide EOP at about less than 1% (w/w). In some aspects, the topical composition comprises ceramide NP, ceramide AP and ceramide EOP at about 3% (w/w). In some aspects, the topical composition comprises ceramide NP, ceramide AP and ceramide EOP wherein each ceramide is present in the topical composition at about less than 1% (w/w).

### Excipients

In some aspects, the topical compositions disclosed herein further comprise at least one excipient. In some aspects, the at least one excipient may comprise any one or more of humectants, surfactant, agents that stabilize emulsion, agents that help form gel, agents that control viscosity, oils, fats, thickening agents, coloring agents, fragrance, or any combination thereof. In some aspects, the excipient that can be used in the composition can be one or more of caprylyl glycol, ethylhexylglycerin glycerin, urea, hyaluronic acid, salicylic acid, alpha hydroxy acids (AHAs) such as glycolic acid and lactic acid, propylene glycol, honey, sorbitol, beeswax, olive oil, coconut oil, lanolin oil (isolanolin), petrolatum (petroleum jelly), mineral oil, glycerin, methyl glyceth, zinc oxide, goat milk, butyl stearate, diglycol laurate, C12-15 alkyl benzoate, aloe vera palmitate, butylene glycol, ethylhexyl palmitate, isododecane, isohexadecane, isopropyl myristate, octyldodecanol, PEG-40 hydrogenated castor oil, pentylene glycol BIO, (1, 3) propanediol, allantoin, lecithin, caprylic capric triglyceride, octyl deodecanol, cetearyl alcohol, squalene, squalene, tocopheryl acetate, polyglyceryl-6 laurate, ceteareth-20, dimethicone, PEG-100 stearate, octyldodecanol, caprylyl glycol, dicaprylyl carbonate, myristyl myristate, hectorite gel, stearyl palmitate, polyamide 3, sorbitol, polyhydroxystearic acid, lauryl laurate, cyclopentasiloxane (silicone gel), glyceryl stearate, sorbitol olivate, Jojoba esters, polysorabate 60, laureth-4, potassium cetyl sulfate, cetyl alcohol, cetearyl alcohol, stearyl alcohol, glyceryl stearate, polyglyceryl-6 laurate, ceteareth-20, sodium lauryl lactylate, myristyl myristate, carbomer, polysorbate 80, pola wax, sorbitan stearate, gum arabic, brassica alcohol, carbomer 980 QD, sodium stearate, polyhydroxystearic acid, PEG-150 distearate, glyceryl oleate, emulsifying wax, glyceryl monooleate, phospholipids, polyoxyethylene alkyl ethers, polyoxyethylene, castor oil derivatives, sorbitan fatty acid esters, polyoxyethylene stearates, polyoxylglycerides, polysorbate, sorbitan esters, sorbitan olivate, hexylene glycol, carbopol 980, cetyl alcohol, stearic acid, carnauba wax, hydroxyethyl cellulose, guar gum, xanthan gum, gelatin, magnesium aluminum silicate, silica, bentonite, cetyl palmitate, ammonium acryloyldimethyltaurate, cetearyl alcohol, glucose-D, hectorite gel, stearyl palmitate, gum arabic, hydroxypropyl starch phosphate, tapioca starch, acrylates octylacrylamide copolymer, carbomer 940, polyamide 3, castor wax, hydroxypropyl methylcellulose, Caesalpinia spinosa gum, brassica alcohol, sodium stearate, polyhydroxystearic acid, tribehenin, arrowroot starch, rice starch, candelilla wax, beeswax, ozokerite wax, sunflower wax, PEG-150 distearate, polyacrylate crosspolymer-6, acrylates C10-30 alkyl acrylate cross polymer, hydroxypropyl guar, cyclopentasiloxane and (silicone gel), glycerin, propanediol, butylene glycol, ethoxydiglycol, isododecane, isohexadecane, octyldodecanol, 1,2-hexane diol, dicaprylyl carbonate, or any combination thereof.

In some aspects, at least one excipient is present in the topical composition at about 0.01% (w/w) to about 90% (w/w). In some aspects of the topical composition, the excipients comprise one or more of polysorbate 20, cetearyl alcohol, glycerin, sodium lauroyl lactylate, carbomer, xanthan gum, or any combination thereof. In some aspects, the topical composition comprises polysorbate 20, cetearyl alcohol, glycerin, sodium lauroyl lactylate, carbomer, and xanthan gum. In some aspects, the disclosed topical composition comprises one or more of polysorbate 20, cetearyl alcohol, glycerin, sodium lauroyl lactylate, carbomer, xanthan gum, or any combination thereof, at about 0.01% (w/w) to about 90% (w/w). In some aspects, the disclosed topical composition comprises polysorbate 20, cetearyl alcohol, glycerin, sodium lauroyl lactylate, carbomer, and xanthan gum, at about 0.01% (w/w) to about 90% (w/w).

In some aspects, the disclosed topical composition comprises polysorbate 20. In some aspects, the topical composition comprises about 0.01% (w/w) to about 15% (w/w) of polysorbate 20. In some aspects, the disclosed composition comprises polysorbate 20. In some aspects, the topical composition comprises about 1% (w/w) to about 10% (w/w) of polysorbate 20. In some aspects, the topical composition comprises polysorbate 20 in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), 7% (w/w), 8% (w/w), 9% (w/w), 10% (w/w), 11% (w/w), 12% (w/w), 13% (w/w), 14% (w/w), or 15% (w/w) of polysorbate 20. In some aspects, the topical composition comprises polysorbate 20 at about 1% (w/w). In some aspects, the topical composition comprises polysorbate 20 at about 10% (w/w).

In some aspects, the disclosed topical composition comprises cetearyl alcohol. In some aspects, the topical composition comprises about 0.01% (w/w) to about 15% (w/w) of cetearyl alcohol. In some aspects, the disclosed composition comprises cetearyl alcohol. In some aspects, the topical composition comprises about 1% (w/w) to about 10% (w/w) of cetearyl alcohol. In some aspects, the topical composition comprises cetearyl alcohol in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), 7% (w/w), 8% (w/w), 9% (w/w), 10% (w/w), 11% (w/w), 12% (w/w), 13% (w/w), 14% (w/w), or 15% (w/w) of cetearyl alcohol. In some aspects, the topical composition comprises cetearyl alcohol at about 1% (w/w). In some aspects, the topical composition comprises cetearyl alcohol at about 10% (w/w).

In some aspects, the disclosed topical composition comprises glycerin. In some aspects, the disclosed composition comprises at about 0.01% (w/w) to about 1% (w/w) of glycerin. In some aspects, the topical composition comprises glycerin in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w) of glycerin. In some aspects, glycerin is present in the topical composition at about 0.01% (w/w). In some aspects, glycerin is present in the topical composition at about less than 1% (w/w).

In some aspects, the disclosed topical composition comprises sodium lauroyl lactylate. In some aspects, the disclosed composition comprises at about 0.01% (w/w) to about 1% (w/w) of sodium lauroyl lactylate. In some aspects, the topical composition comprises sodium lauroyl lactylate in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w) of sodium lauroyl lactylate. In some aspects, sodium lauroyl lactylate is present in the topical composition at about 0.01% (w/w). In some aspects, sodium lauroyl lactylate is present in the topical composition at about less than 1% (w/w).

In some aspects, the disclosed composition comprises carbomer. In some aspects, the disclosed composition comprises at about 0.01% (w/w) to about 1% (w/w) of carbomer. In some aspects, the topical composition comprises carbomer in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w) of carbomer. In some aspects, carbomer is present in the topical composition at about 0.01% (w/w). In some aspects, carbomer is present in the topical composition at about less than 1% (w/w).

In some aspects, the disclosed topical composition comprises xanthan gum. In some aspects, the topical composition comprises about 0.01% (w/w) to about 1% (w/w) of xanthan gum. In some aspects, the topical composition comprises xanthan gum in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w) of xanthan gum. In some aspects, xanthan gum is present in the topical composition at about 0.01% (w/w). In some aspects, xanthan gum is present in the topical composition at about less than 1% (w/w).

### Solvents

In some aspects, the disclosed topical composition comprises at least one solvent. In some aspects, the solvent is one or more of water, ethanol, aliphatic alcohols, esters, ethers, ketones, amines and hydrocarbons. In some aspects, the solvent is water.

In some aspects, the topical composition comprises water as a solvent. In some aspects, the topical composition comprises about 60% (w/w) to about 90% (w/w), or about 70% (w/w) to about 80% (w/w), or about 84% (w/w) to about 86% (w/w), or about 86% (w/w) to about 88% (w/w), or about 88% (w/w) to about 90% (w/w) of water. In some aspects, the composition comprises about 60% (w/w), about 65% (w/w), about 70% (w/w), about 75% (w/w), about 80% (w/w), about 81% (w/w), about 82% (w/w), about 83% (w/w), about 84% (w/w), about 85% (w/w), about 86% (w/w), about 87% (w/w), about 88% (w/w), about 89% (w/w), or about 90% (w/w) of water.

### Skin Conditioner and Emollient

In some aspects, the topical composition disclosed herein, further comprises at least one skin conditioner and/or at least one emollient. In some aspects, the topical composition comprises at least one skin conditioner and/or at least one emollient wherein the skin conditioner and/or emollient is one or more of caprylic/capric triglyceride, coconut alkanes, glyceryl stearate, Helianthus annuus seed oil, Aloe barbadensis leaf juice, Cocos nucifera oil, coco-caprylate/caprate, phytosphingosine, cholesterol, ethylhexylglycerin, or any combination thereof. In some aspects, the topical composition comprises caprylic/capric triglyceride, coconut alkanes, glyceryl stearate, Helianthus annuus seed oil, Aloe barbadensis leaf juice, Cocos nucifera oil, coco-caprylate/caprate, phytosphingosine, cholesterol, and ethylhexylglycerin.

In some aspects, at least one skin conditioner and/or at least one emollient is present in the composition at about 0.01% (w/w) to about 15% (w/w). In some aspects, one or more of caprylic/capric triglyceride, coconut alkanes, glyceryl stearate, Helianthus annuus seed oil, Aloe barbadensis leaf juice, Cocos nucifera oil, coco-caprylate/caprate, phytosphingosine, cholesterol, ethylhexylglycerin, or any combination thereof, is present in the composition at about 0.01% (w/w) to about 15% (w/w). In some aspects, the topical composition comprises caprylic/capric triglyceride, coconut alkanes, glyceryl stearate, Helianthus annuus seed oil, Aloe barbadensis leaf juice, Cocos nucifera oil, coco-caprylate/caprate, phytosphingosine, cholesterol, and ethylhexylglycerin wherein each of the caprylic/capric triglyceride, coconut alkanes, glyceryl stearate, Helianthus annuus seed oil, Aloe barbadensis leaf juice, Cocos nucifera oil, coco-caprylate/caprate, phytosphingosine, cholesterol, and ethylhexylglycerin is present at about 0.01% (w/w) to about 15% (w/w).

In some aspects, the disclosed composition comprises caprylic/capric triglyceride. In some aspects, the topical composition comprises about 0.01% (w/w) to about 15% (w/w) of caprylic/capric triglyceride. In some aspects, the disclosed composition comprises caprylic/capric triglyceride. In some aspects, the topical composition comprises about 1% (w/w) to about 10% (w/w) of caprylic/capric triglyceride. In some aspects, the topical composition comprises caprylic/capric triglyceride in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), 7% (w/w), 8% (w/w), 9% (w/w), 10% (w/w), 11% (w/w), 12% (w/w), 13% (w/w), 14% (w/w), or 15% (w/w) of caprylic/capric triglyceride. In some aspects, the topical composition comprises caprylic/capric triglyceride at about 1% (w/w). In some aspects, the topical composition comprises caprylic/capric triglyceride at about 10% (w/w).

In some aspects, the disclosed composition comprises coconut alkanes. In some aspects, the topical composition comprises about 0.01% (w/w) to about 15% (w/w) of coconut alkanes. In some aspects, the disclosed composition comprises coconut alkanes. In some aspects, the topical composition comprises about 1% (w/w) to about 10% (w/w) of coconut alkanes. In some aspects, the topical composition comprises coconut alkanes in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), 7% (w/w), 8% (w/w), 9% (w/w), 10% (w/w), 11% (w/w), 12% (w/w), 13% (w/w), 14% (w/w), or 15% (w/w) of coconut alkanes. In some aspects, the topical composition comprises coconut alkanes at about 1% (w/w). In some aspects, the topical composition comprises coconut alkanes at about 10% (w/w).

In some aspects, the disclosed composition comprises glyceryl stearate. In some aspects, the topical composition comprises about 0.01% (w/w) to about 15% (w/w) glyceryl stearate. In some aspects, the disclosed composition comprises glyceryl stearate. In some aspects, the topical composition comprises about 1% (w/w) to about 10% (w/w) of glyceryl stearate. In some aspects, the topical composition comprises glyceryl stearate in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), 7% (w/w), 8% (w/w), 9% (w/w), 10% (w/w), 11% (w/w), 12% (w/w), 13% (w/w), 14% (w/w), or 15% (w/w) of glyceryl stearate. In some aspects, the topical composition comprises glyceryl stearate at about 1% (w/w). In some aspects, the topical composition comprises glyceryl stearate at about 10% (w/w).

In some aspects, the disclosed composition comprises Helianthus annuus seed oil. In some aspects, the topical composition comprises about 0.01% (w/w) to about 15% (w/w) Helianthus annuus seed oil. In some aspects, the disclosed composition comprises Helianthus annuus seed oil. In some aspects, the topical composition comprises about 1% (w/w) to about 10% (w/w) of Helianthus annuus seed oil. In some aspects, the topical composition comprises Helianthus annuus seed oil in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), 7% (w/w), 8% (w/w), 9% (w/w), 10% (w/w), 11% (w/w), 12% (w/w), 13% (w/w), 14% (w/w), or 15% (w/w) of Helianthus annuus seed oil. In some aspects, the topical composition comprises Helianthus annuus seed oil at about 1% (w/w). In some aspects, the topical composition comprises Helianthus annuus seed oil at about 10% (w/w).

In some aspects, the disclosed composition comprises Aloe barbadensis leaf juice. In some aspects, the topical composition comprises about 0.01% (w/w) to about 15% (w/w) Aloe barbadensis leaf juice. In some aspects, the disclosed composition comprises Aloe barbadensis leaf juice. In some aspects, the topical composition comprises about 1% (w/w) to about 10% (w/w) of Aloe barbadensis leaf juice. In some aspects, the topical composition comprises Aloe barbadensis leaf juice in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), 7% (w/w), 8% (w/w), 9% (w/w), 10% (w/w), 11% (w/w), 12% (w/w), 13% (w/w), 14% (w/w), or 15% (w/w) of Aloe barbadensis leaf juice. In some aspects, the topical composition comprises Aloe barbadensis leaf juice at about 1% (w/w). In some aspects, the topical composition comprises Aloe barbadensis leaf juice at about 10% (w/w).

In some aspects, the disclosed composition comprises Cocos nucifera oil. In some aspects, the topical composition comprises about 0.01% (w/w) to about 15% (w/w) Cocos nucifera oil. In some aspects, the disclosed composition comprises Cocos nucifera oil. In some aspects, the topical composition comprises about 1% (w/w) to about 10% (w/w) Cocos nucifera oil. In some aspects, the topical composition comprises Cocos nucifera oil in the amount of about 0.01% (w/w) to about 0.05% (w/w), or about 0.05% (w/w) to about 0.1% (w/w), or about 0.1% (w/w) to about 0.5% (w/w), or about 0.5% (w/w) to about 1% (w/w). In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), 7% (w/w), 8% (w/w), 9% (w/w), 10% (w/w), 11% (w/w), 12% (w/w), 13% (w/w), 14% (w/w), or 15% (w/w) of Cocos nucifera oil. In some aspects, the topical composition comprises Cocos nucifera oil at about 1% (w/w). In some aspects, the topical composition comprises Cocos nucifera oil at about 10% (w/w).

In some aspects, the disclosed composition comprises coco-caprylate/caprate. In some aspects, the topical composition comprises about 0.01% (w/w) to about 1% (w/w) of coco-caprylate/caprate. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of coco-caprylate/caprate. In some aspects, coco-caprylate/caprate is present in the composition at about 0.01% (w/w). In some aspects, coco-caprylate/caprate is present in the composition at about less than 1% (w/w).

In some aspects, the disclosed composition comprises phytosphingosine. In some aspects, the topical composition comprises about 0.01% (w/w) to about 1% (w/w) of coco-phytosphingosine. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of phytosphingosine. In some aspects, phytosphingosine is present in the composition at about 0.01% (w/w). In some aspects, phytosphingosine is present in the composition at about less than 1% (w/w).

In some aspects, the disclosed composition comprises cholesterol. In some aspects, the topical composition comprises about 0.01% (w/w) to about 1% (w/w) of cholesterol. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of cholesterol. In some aspects, cholesterol is present in the composition at about 0.01% (w/w). In some aspects, cholesterol is present in the composition at about less than 1% (w/w).

In some aspects, the disclosed composition comprises ethylhexylglycerin. In some aspects, the topical composition comprises about 0.01% (w/w) to about 1% (w/w) of ethylhexylglycerin. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of ethylhexylglycerin. In some aspects, ethylhexylglycerin is present in the composition at about 0.01% (w/w). In some aspects, ethylhexylglycerin is present in the composition at about less than 1% (w/w).

### Anti-Oxidant

In some aspects, the topical composition disclosed herein, further comprises at least one antioxidant. In some aspects, the topical composition comprises at least one antioxidant wherein the skin conditioner and/or emollient is one or more of gallates, flavonoids, butyl-hydroxytoluene (BHT), butyl-hydroxyanisole (BHA), octadecenedioic acid, hydroxydecanoic acid, mannitol, vitamin C, lysine azelate, rutin, quercetin, plant polyphenolic agent (for e.g, grape, oregano, grape, sage, sunflower, maritime pine bark, rosemary, marjoram, crocus, French saffron, wild carrot, hop, coffee, green coffee, witch hazel, oak, camellia, red clover, flax, ginger, magnolia, edelweiss, burdock, emblica, gingko, lemon, blueberry, apple, cherry, birch, moringa, thyme, cornflower, geranium, white lotus, marshmallow, bilberry, cranberry extract, pomegranate nectar, polygonum, green tea, white tea, soy), vitamin E, carotenoids, or any combination thereof. In some aspects, the topical composition comprises an antioxidant, wherein the antioxidant is tocopheryl acetate.

In some aspects, the disclosed composition comprises tocopheryl acetate. In some aspects, the topical composition comprises about 0.01% (w/w) to about 1% (w/w) of tocopheryl acetate. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of tocopheryl acetate. In some aspects, tocopheryl acetate is present in the composition at about 0.01% (w/w). In some aspects, tocopheryl acetate is present in the composition at about less than 1% (w/w).

### Preservative

In some aspects, the topical composition further comprises at least one preservative. In some aspects, the preservative is one or more of phenoxyethanol, potassium sorbate, or any combination thereof. In some aspects, the topical composition comprises phenoxyethanol. In some aspects, the preservative is present in the composition at about 0.01% (w/w) to about 1% (w/w). In some aspects, the topical composition comprises potassium sorbate, at about 0.01% (w/w) to about 1% (w/w).

In some aspects, the topical composition comprises phenoxyethanol. In some aspects, the topical composition comprises about 0.01% (w/w) to about 1% (w/w) of phenoxyethanol. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of phenoxyethanol. In some aspects, phenoxyethanol is present in the composition at about 0.01% (w/w). In some aspects, phenoxyethanol is present in the composition at about less than 1% (w/w).

In some aspects, the topical composition comprises potassium sorbate. In some aspects, the topical composition comprises about 0.01% (w/w) to about 1% (w/w) of potassium sorbate. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of potassium sorbate. In some aspects, potassium sorbate is present in the composition at about 0.01% (w/w). In some aspects, potassium sorbate is present in the composition at about less than 1% (w/w).

### Buffer

In some aspects, the topical composition further comprises at least one buffer. In some aspects, the buffer is sodium hydroxide. In some aspects, the buffer is present in the composition at about 0.01% (w/w) to about 1% (w/w).

In some aspects, the topical composition comprises sodium hydroxide. In some aspects, the topical composition comprises about 0.01% (w/w) to about 1% (w/w) of sodium hydroxide. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of sodium hydroxide. In some aspects, sodium hydroxide is present in the composition at about 0.01% (w/w). In some aspects, sodium hydroxide is present in the composition at about less than 1% (w/w).

### Chelator

In some aspects, the topical composition further comprises at least one chelator. In some aspects, the chelator is sodium phytate. In some aspects, the chelator is present in the composition at about 0.01% (w/w) to about 1% (w/w).

In some aspects, the topical composition comprises sodium phytate. In some aspects, the topical composition comprises about 0.01% (w/w) to about 1% (w/w) of sodium phytate. In some aspects, the topical composition comprises about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or 1% (w/w) of sodium phytate. In some aspects, sodium phytate is present in the composition at about 0.01% (w/w). In some aspects, sodium phytate is present in the composition at about less than 1% (w/w).

In some aspects, the composition further comprise oils, fats, thickening agents, coloring agents, fragrance, or any combination thereof. Such ingredients may be present in the compositions, at about 0.01% (w/w) to about 1% (w/w). In further aspects, the present composition may include one or more of other active and inactive ingredients, including, but not limited to vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, antiacne agents, anesthetics, antiseptics, insect repellents, skin cooling compounds, skin protectants, skin penetration enhancers, exfoliants, lubricants, fragrances, colorants, staining agents, depigmenting agents, hypopigmenting agents, preservatives, stabilizers, pharmaceutical agents, photostabilizing agents, and mixtures thereof. In addition to the foregoing, the composition of the disclosure may contain any other compound for the treatment of skin disorders.

In some aspects, the disclosed topical composition comprises water, caprylic/capric triglyceride, coconut alkanes, glyceryl stearate, polysorbate 20, Helianthus annuus seed oil, Aloe barbadensis leaf juice, cetearyl alcohol, Cocos nucifera oil, glycerin, coco-caprylate/caprate, sodium phytate, Chlorella vulgaris extract, vegan collagen peptide (for e.g., sh-polypeptide-121), ceramide NP, ceramide AP, ceramide EOP, phytosphingosine, cholesterol, sodium lauroyl lactylate, carbomer, ethylhexylglycerin, xanthan gum, phenoxyethanol, sodium hydroxide, tocopheryl acetate, potassium sorbate, or any combination thereof. In some aspects, the topical composition comprises one or more ingredients at range of amounts listed in Table 1.

TABLE 1
Topical composition

| Ingredients | Amount % (w/w) |
|---|---|
| Water | about 1% to about 90% |
| Caprylic/capric triglyceride | about 1% to about 10% |
| Coconut alkanes | about 1% to about 10% |
| Glyceryl stearate | about 1% to about 10% |
| Polysorbate 20 | about 1% to about 10% |
| Helianthus annuus seed oil | about 1% to about 10% |
| Aloe barbadensis leaf juice | about 1% to about 10% |
| Cetearyl alcohol | about 1% to about 10% |
| Cocos nucifera oil | about 1% to about 10% |
| Glycerin | about 0.01% to about 1% |
| Coco-caprylate/caprate | about 0.01% to about 1% |
| Sodium phytate | about 0.01% to about 1% |
| Chlorella vulgaris extract | about 0.01% to about 1% |
| Vegan collagen peptide (sh-polpeptide-121) | about 0.01% to about 1% |
| Ceramide NP | about 0.01% to about 1% |
| Ceramide AP | about 0.01% to about 1% |
| Ceramide EOP | about 0.01% to about 1% |
| Phytosphingosine | about 0.01% to about 1% |
| Cholesterol | about 0.01% to about 1% |
| Sodium lauroyl lactylate | about 0.01% to about 1% |
| Carbomer | about 0.01% to about 1% |
| Ethylhexylglycerin | about 0.01% to about 1% |
| Xanthan gum | about 0.01% to about 1% |
| Phenoxylethanol | about 0.01% to about 1% |
| Sodium hydroxide | about 0.01% to about 1% |
| Tocopheryl acetate | about 0.01% to about 1% |
| Potassium sorbate | about 0.01% to about 1% |

In some aspects, the compositions disclosed herein may be in any suitable form including but not limited to solids, liquids (for e.g., lotion), suspensions, semisolids (such as creams, gels, pastes or “sticks”), powders, or finely dispersed liquids such as sprays or mists. In some aspects, the compositions may be packaged into bottles, tubes, tablets, applicators, capsules, soap, facial peel, or facial mask. In certain aspects, the composition is formulated as a cosmetic composition non-limiting examples of which include serum, solution, suspension, lotion, oil, cream, gel, toner, stick, pencil, ointment, paste, foam, powder, balm, mousse, shaving cream, wipe, strip, patch, wound dressing and adhesive bandage, hydrogel, film-forming product, facial and skin mask, cosmetics (e.g., foundations, eye liners, eye shadows, concealer, primer), and the like. In certain aspects, the composition of the present disclosure is formulated as a lotion. The composition formulated as a lotion can be a composition disclosed in any one of the Tables 1-5. In one specific aspect, the topical composition is formulated as a lotion for use during day. In some aspects, the lotion composition of the present disclosure is formulated as a stable emulsion. The emulsion can be an oil-in-water emulsion or a water-in-oil emulsion.

Also contemplated are containers comprising a composition of the present disclosure. In non-limiting aspects, the container can be a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The containers can optionally have spray, pump, or squeeze mechanisms.

The present composition may be prepared into a desired formulation by any known methods in the art. For example, the composition may be prepared by mixing one or more disclosed ingredients into a homogeneous product. The method comprises mixing the vegan collagen peptide, Chlorella vulgaris, fruit extract, ceramide optionally one or more additional ingredients as described herein, and water to form the skin care formulation. The mixing may be carried out, for example, by using a mixer or blender.

### II. Methods of Using

In some aspects, the present disclosure also encompasses methods of using the compositions disclosed herein for improving one or more condition of the skin in a subject.

The method of improving one or more condition of the skin of a subject comprises applying a composition of the present disclosure to the skin of the subject. In some aspects, the composition is applied for about 24 hours, about 20 hours, about 16 hours, about 12 hours, about 10 hours, about 8 hours, about 6 hours, about 4 hours, about 2 hours, about 1 hour or less. In further aspects, the composition is applied at least three times, at least four times, at least five times, at least six times, at least seven times, at least eight times, at least nine times, or at least ten times over a period of time (e.g., one week, two weeks, three weeks, a month, two months, three months, etc.). The method may include applying the composition a plurality of times (e.g., one, two, three, four, five, or six times) each day for a period of time (e.g., one week, two weeks, three weeks, a month, two months, three months, etc.).

In certain aspects, the method may include applying an amount of the composition such that about 0.01 gram or more is applied to the skin. For example, the composition may be applied such that about 0.01 gram per decimeter (g/dm²) or more, about 0.5 g/dm² or more, about 07.5 g/dm² or more, about 1 g/dm² or more, about 1.5 g/dm² or more, about 2 g/dm² or more, about 2.5 g/dm² or more, or about 3 g/dm² or of the composition is applied to the skin. In some cases, the composition is applied such that about 0.1 to about 10 gram per decimeter (g/dm²), about 0.1 to about 9 g/dm², about 0.1 to about 8 g/dm², about 0.1 to about 7 g/dm², about 0.1 to about 6 g/dm², about 0.1 to about 5 g/dm², about 0.1 to about 4 g/dm², about 0.1 to about 3 g/dm², about 0.1 to about 2 g/dm², about 0.1 to about 1 g/dm²; about 0.5 to about 10 gram per decimeter (g/dm²), about 0.5 to about 9 g/dm², about 0.5 to about 8 g/dm², about 0.5 to about 7 g/dm², about 0.5 to about 6 g/dm², about 0.5 to about 5 g/dm², about 0.5 to about 4 g/dm², about 0.5 to about 3 g/dm², about 0.5 to about 2 g/dm², about 0.5 to about 1 g/dm²; or about 1 to about 10 gram per decimeter (g/dm²), about 1 to about 9 g/dm², about 1 to about 8 g/dm², about 1 to about 7 g/dm², about 1 to about 6 g/dm², about 1 to about 5 g/dm², about 1 to about 4 g/dm², about 1 to about 3 g/dm², or about 1 to about 2 g/dm² of the composition is applied to the skin.

In some aspects, the compositions disclosed herein is administered to the skin of a subject to prevent, reduce, or treat fine lines, aging of the skin cells, crow's feet, wrinkles, moisture loss, hyperpigmentation, uneven skin tone, under eye circles, uneven skin pigmentation, crepe skin, collagen loss, elastin loss, oxidation, skin redness, roughness, free radical formation, UV damage, photo-damage, blue light damage, or any combination thereof. In one aspect, the administration of the composition disclosed herein reduce one or more of fine lines, aging of the skin cells, crow's feet, wrinkles, moisture loss, hyperpigmentation, uneven skin tone, under eye circles, uneven skin pigmentation, crepe skin, collagen loss, elastin loss, oxidation, skin redness, roughness, free radical formation, UV damage, photo-damage, and blue light damage in the skin of the subject, by at least 10%, e.g., 20%, 30%, 40%, 50%, 60%, 70%, 80%, 90%, 95% or above, as compared to the subject before administration of the composition or a control subject not receiving the composition. The change in the skin condition can be assessed by one or more a skin care professionals, or can be undertaken by self-assessment, using, for example, image-based analysis. Images, for example facial pictures, can be taken before and after treatments, or of the control subjects, and visually compared to read the differences in skin condition. In an alternate aspect, photographs may be analyzed by a computer, using a computer-based grading using a facial computational algorithm that have been previously validated for assessment of the skin condition.

In some aspects, the disclosed topical composition is administered to the skin of a subject to enhance collagen production, elastin production, skin immunity, hydration, clarity, radiance, passage for absorption, skin brightness, healthy glow, even tone, elasticity, firmness, smoothness, or any combination thereof. In one aspect, the administration of the composition disclosed herein enhance one or more of collagen production, elastin production, skin immunity, hydration, clarity, radiance, passage for absorption, skin brightness, healthy glow, even tone, elasticity, firmness, and smoothness in the skin of the subject, by at least 10%, e.g., 20%, 30%, 40%, 50%, 60%, 70%, 80%, 90%, 95% or above, as compared to the subject before administration of the composition or a control subject not receiving the composition. The change in the skin condition can be assessed by one or more skin care professionals, or can be undertaken by self-assessment, using, for example, image-based analysis. Images, for example facial pictures, can be taken before and after treatments, or of the control subjects, and visually compared to read the differences in skin condition. In an alternate aspect, photographs may be analyzed by a computer, using a computer-based grading using a facial computational algorithm that have been previously validated for assessment of the skin condition.

In some aspects, the composition may be applied to the skin one or more times daily, one or more times weekly, one or more time monthly or at any interval for any duration of time. For example, as a liquid composition, 3-5 drops of the composition may be used daily or multiple times in a day. By way of another example, as a cream or lotion composition, a thin, medium, or thick layer of the composition may be used daily or multiple times in a day. In some instances, the composition is a cream and a thin layer is used. In some aspects, such use may provide reduction in visible fine lines, wrinkles, hyperpigmentation, uneven pigmentation, crow's feet, skin redness, roughness, and/or under-eye circles by about 1% to 20% as estimated by methods known in the art. In some aspects, use of 1, or 2, or 3, or 4, or 5, or 6, or 7, or 8, or 9, or 10 drops daily can reduce one or more of fine lines, aging of the skin cells, crow's feet, wrinkles, moisture loss, hyperpigmentation, uneven skin tone, under eye circles, uneven skin pigmentation, crepe skin, collagen loss, elastin loss, oxidation, skin redness, roughness, free radical formation, UV damage, photo-damage, blue light damage. In some aspects, the disclosed topical composition is administered to the skin of a subject to enhance collagen production, elastin production, skin immunity, hydration, clarity, radiance, passage for absorption, skin brightness, healthy glow, even tone, elasticity, firmness, smoothness, by about 1% to 20% as estimated by methods known in the art.

In some aspects, the composition can be applied to any external or exposed part of the body. For example, the composition can be applied to the face, hands, elbow, knees, legs, toes, neck, or fingers. In some aspects, the composition or lotion can be used on skin around the eye, face, neck, hands and other targeted areas to increase skin firmness and lighten under-eye circles.

In some aspects, the subject is a human. In some aspects, the subject is a human of any age from 0-120 yrs.

### III. Kits

In some aspects, the current disclosure also encompasses kits comprising the topical composition disclosed herein and instructions for use. The kit may include a container comprising the composition disclosed herein. The container can be a bottle, a dispenser, a package, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The container can dispense a pre-determined amount of the composition. In certain aspects, the compositions is dispensed in a spray, dollop, or liquid. The container can include indicia on its surface. The indicia can be a word, an abbreviation, a picture, or a symbol.

The container can dispense a pre-determined amount of the composition. In other aspects, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### I. Terminology

The phraseology and terminology employed herein are for the purpose of description and should not be regarded as limiting. For example, the use of a singular term, such as, “a” is not intended as limiting of the number of items.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, “and/or” refers to and encompasses any and all possible combinations of one or more of the associated listed items, as well as the lack of combinations where interpreted in the alternative (“or”).

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “topical composition” or “topical formulation” or “topical cosmetics” or “topical products” as used herein encompasses substances that may be applied topically to the skin or mucous membranes of humans or animals in order to alter the subject's appearance, to protect the subject from the environment, or to produce a biological change in the skin or other tissue for therapeutic, preventive or cosmetic purposes. Topical products occur in a variety of forms, including solids, liquids, suspensions, semisolids (such as creams, gels, pastes, or “sticks”), powders, or finely dispersed liquids such as sprays or mists. Examples of topical products commonly classified as “cosmetics” include skin care products such as creams, lotions, moisturizers, serums, and “treatment cosmetics” such as exfoliants and/or skin cell renewal agents.

As used herein, “individual”, “subject”, “host”, and “patient” can be used interchangeably herein and refer to any mammalian subject, for example, humans.

The term “emollient” as used herein encompasses ingredients in skin creams, lotions, moisturizers or ointments that form a film on the skin. These ingredients can relieve dryness, itching, and scaling. Emollients can help the skin feel more comfortable if you have eczema, psoriasis, dry, or sensitive skin.

The term “humectant” as used herein includes a substance that draws water into the skin, hair, or nails. In the skin, this may come from the deeper layers, or from the air if it is humid enough. Humectants are useful for adding hydration without feeling heavy or oily. Humectants include ingredients such as glycerin, urea, AHAs, and hyaluronic acid.

### Examples

The following examples are included to demonstrate some aspects of the disclosure. However, those of skill in the art should, in light of the present disclosure, appreciate that many changes can be made in the specific aspects which are disclosed and still obtain a like or similar result without departing from the spirit and scope of the present disclosure.

Example of Topical Formulation I is provided in Table 2.

TABLE 2
Topical Formulation I

| Ingredients | Amount % (w/w) |
|---|---|
| Water | about 80% to about 90% |
| Vegan collagen peptide | about 0.01% to about 1% |
| Chlorella vulgaris | about 0.01% to about 1% |
| Ceramide | about 0.01% to about 1% |
| Excipient | about 0.1% to about 10% |

Table 3 provides an example of topical formulation II according to one aspect of the disclosure.

TABLE 3
Topical formulation II

| Ingredients | Amount % (w/w) |
|---|---|
| Water | about 80% to about 90% |
| Sh-Polypeitde-121 | about 0.01% to about 1% |
| Chlorella vulgaris | about 0.01% to about 1% |
| Ceramide NP | about 0.01% to about 1% |
| Ceramide AP | about 0.01% to about 1% |
| Ceramide EOP | about 0.01% to about 1% |
| Excipient | about 0.1% to about 10% |

Table 4 further provides an example of a topical formulation Ill according to another aspect of the disclosure.

TABLE 4
Topical Formulation III

| Ingredients | Amount % (w/w) |
|---|---|
| Water | about 80% to about 90% |
| Caprylic/capric triglyceride | about 1% to about 10% |
| Coconut alkanes | about 1% to about 10% |
| Glyceryl stearate | about 1% to about 10% |
| Polysorbate 20 | about 1% to about 10% |
| Helianthus annuus seed oil | about 1% to about 10% |
| Aloe barbadensis leaf juice | about 1% to about 10% |
| Cetearyl alcohol | about 1% to about 10% |
| Cocos nucifera oil | about 1% to about 10% |
| Glycerin | about 0.01% to about 1% |
| Coco-caprylate/caprate | about 0.01% to about 1% |
| Sodium phytate | about 0.01% to about 1% |
| Chlorella vulgaris extract | about 0.01% to about 1% |
| Sh-polpeptide-121) | about 0.01% to about 1% |
| Ceramide NP | about 0.01% to about 1% |
| Ceramide AP | about 0.01% to about 1% |
| Ceramide EOP | about 0.01% to about 1% |
| phytosphingosine | about 0.01% to about 1% |
| Cholesterol | about 0.01% to about 1% |
| Sodium lauroyl lactylate | about 0.01% to about 1% |
| Carbomer | about 0.01% to about 1% |
| Ethylhexylglycerin | about 0.01% to about 1% |
| Xanthan gum | about 0.01% to about 1% |
| Phenoxylethanol | about 0.01% to about 1% |
| Sodium hydroxide | about 0.01% to about 1% |
| Tocopheryl acetate | about 0.01% to about 1% |
| Potassium sorbate | about 0.01% to about 1% |

Table 5 further provides an example of a topical formulation IV according to another aspect of the disclosure.

TABLE 5
Topical Formulation IV

| Ingredients | Amount % (w/w) |
|---|---|
| Water | about 80% to about 90% |
| Caprylic/capric triglyceride | about 1% to about 10% |
| Coconut alkanes | about 1% to about 10% |
| Glyceryl stearate | about 1% to about 10% |
| Polysorbate 20 | about 1% to about 10% |
| Helianthus annuus seed oil | about 1% to about 10% |
| Aloe barbadensis leaf juice | about 1% to about 10% |
| Cetearyl alcohol | about 1% to about 10% |
| Cocos nucifera oil | about 1% to about 10% |
| Glycerin | about less than 1% |
| Coco-caprylate/caprate | about less than 1% |
| Sodium phytate | about less than 1% |
| Chlorella vulgaris extract | about less than 1% |
| Vegan collagen peptide (sh-polpeptide-121) | about less than 1% |
| Ceramide NP | about less than 1% |
| Ceramide AP | about less than 1% |
| Ceramide EOP | about less than 1% |
| phytosphingosine | about less than 1% |
| Cholesterol | about less than 1% |
| Sodium lauroyl lactylate | about less than 1% |
| Carbomer | about less than 1% |
| Ethylhexylglycerin | about less than 1% |
| Xanthan gum | about less than 1% |
| Phenoxylethanol | about less than 1% |
| Sodium hydroxide | about less than 1% |
| Tocopheryl acetate | about less than 1% |
| Potassium sorbate | about less than 1% |

What is claimed is:

1. A topical composition, comprising:
a. vegan collagen peptide;
b. Chlorella vulgaris extract;
c. at least one ceramide;
d. at least one excipient; and
e. at least one solvent.

2. The topical composition of claim 1, wherein the vegan collagen peptide is present in the composition at a range of about 0.01% (w/w) to about 1% (w/w).

3. The topical composition of claim 2, wherein the vegan collagen peptide is Sh-Polypeptide-121.

4. The topical composition of claim 1, wherein the Chlorella vulgaris extract is present in the composition at a range of about 0.01% (w/w) to about 1% (w/w).

5. The topical composition of claim 1, wherein at least one ceramide is Ceramide NP, Ceramide AP, Ceramide EOP, or any combination thereof.

6. The topical composition of claim 5, wherein the ceramide is present in the composition at a range of about 0.01% (w/w) to about 3% (w/w).

7. The topical composition of claim 1, wherein the at least one excipient or solvent is present in the composition at about 0.1% (w/w) to about 90% (w/w).

8. The topical composition of claim 7, wherein the at least one excipient is polysorbate 20, cetearyl alcohol, glycerin, sodium lauroyl lactylate, carbomer, xanthan gum, or any combination thereof.

9. The topical composition of claim 7, wherein the at least one solvent is water.

10. The topical composition of claim 1, wherein the composition further comprises at least one skin conditioner and/or at least one emollient selected from caprylic/capric triglyceride, coconut alkanes, glyceryl stearate, Helianthus annuus seed oil, Aloe barbadensis leaf juice, Cocos nucifera oil, coco-caprylate/caprate, phytosphingosine, cholesterol, ethylhexylglycerin, or any combination thereof.

11. The topical composition of claim 10, wherein the at least one skin conditioner and/or at least one emollient is present in the composition at about 1% (w/w) to about 10% (w/w).

12. The topical composition of claim 1, wherein the composition further comprises an antioxidant, wherein the antioxidant is Tocopheryl acetate.

13. The topical composition of claim 12, wherein the antioxidant is present at the composition at about 0.01% (w/w) to about 1% (w/w).

14. The topical composition of any one of the preceding claims, wherein the composition further comprises at least one preservative selected from phenoxyethanol, potassium sorbate, or any combination thereof.

15. The topical composition of claim 14, wherein the at least one preservative is present in the composition at about 0.01% (w/w) to about 1% (w/w).

16. The topical composition of claim 1, wherein the composition further comprises a buffer, wherein the buffer is sodium hydroxide.

17. The topical composition of claim 16, wherein the at least one buffer is present in the composition at about 0.01% (w/w) to about 1% (w/w).

18. The topical composition of claim 1, wherein the composition further comprises a chelator, wherein the chelator is sodium phytate.

19. The topical composition of claim 18, wherein the chelator is present in the composition at about 0.01% (w/w) to about 1% (w/w).

20. A topical composition comprising: water, caprylic/capric triglyceride, coconut alkanes, glyceryl stearate, polysorbate 20, Helianthus annuus seed oil, Aloe barbadensis leaf juice, cetearyl alcohol, Cocos nucifera oil, glycerin, coco-caprylate/caprate, sodium phytate, Chlorella vulgaris extract, vegan collagen peptide, ceramide NP, ceramide AP, ceramide EOP, phytosphingosine, cholesterol, sodium lauroyl lactylate, carbomer, ethylhexylglycerin, xanthan gum, phenoxyethanol, sodium hydroxide, tocopheryl acetate, potassium sorbate, or any combination thereof.

21. A topical composition comprising:
Water: about 80% (w/w) to about 90% (w/w);
Caprylic/Capric triglyceride: about 1% (w/w) to about 10% (w/w);
Coconut alkanes: about 1% (w/w) to about 10% (w/w);
Glyceryl stearate: about 1% (w/w) to about 10% (w/w);
Polysorbate 20: about 1% (w/w) to about 10% (w/w);
Helianthus annuus seed oil: about 1% (w/w) to about 10% (w/w);
Aloe barbadensis leaf juice: about 1% (w/w) to about 10% (w/w);
Cetaryl alcohol: about 1% (w/w) to about 10% (w/w);
Cocos nucifera oil: about 1% (w/w) to about 10% (w/w);
Glycerin: about 0.01% (w/w) to about 1% (w/w);
Coco-Caprylate/Caprate: about 0.01% (w/w) to about 1% (w/w);
Sodium Phytate: about 0.01% (w/w) to about 1% (w/w);
Chlorella vulgaris extract: about 0.01% (w/w) to about 1% (w/w);
Sh-Polypeptide-121: about 0.01% (w/w) to about 1% (w/w);
Ceramide NP: about 0.01% (w/w) to about 1% (w/w);
Ceramide AP: about 0.01% (w/w) to about 1% (w/w);
Ceramide EOP: about 0.01% (w/w) to about 1% (w/w);
Phytosphingosine: about 0.01% (w/w) to about 1% (w/w);
Cholesterol: about 0.01% (w/w) to about 1% (w/w);
Sodium lauroyl lactylate: about 0.01% (w/w) to about 1% (w/w);
Carbomer: about 0.01% (w/w) to about 1% (w/w);
Ethylhexylglycerin: about 0.01% (w/w) to about 1% (w/w);
Xanthan gum: about 0.01% (w/w) to about 1% (w/w);
Phenoxyethanol: about 0.01% (w/w) to about 1% (w/w);
Sodium hydroxide: about 0.01% (w/w) to about 1% (w/w);
Tocopheryl acetate: about 0.01% (w/w) to about 1% (w/w);
Potassium sorbate: about 0.01% (w/w) to about 1% (w/w).

22. The topical composition of claim 21, wherein the composition is in the form of a lotion.

23. A method of improving the condition of the skin of a subject in need thereof comprising: applying to the skin of the subject the topical composition of claim 21; wherein applying the topical composition: (a) prevent, reduce, or treat fine lines, aging of the skin cells, crow's feet, wrinkles, moisture loss, hyperpigmentation, uneven skin tone, under eye circles, uneven skin pigmentation, crepe skin, collagen loss, elastin loss, oxidation, skin redness, roughness, free radical formation, UV damage, photo-damage, blue light damage, or any combination thereof; and/or (b) enhance collagen production, elastin production, skin immunity, hydration, clarity, radiance, passage for absorption, skin brightness, healthy glow, even tone, elasticity, firmness, smoothness, or any combination thereof.
